(19) United States
(12) Patent Application Publication
(10) Pub. No.: US 2015/0051543 A1
(43) Pub. Date: Feb. 19, 2015

(54) HIGH-PRESSURE INFLATION DEVICES AND METHODS OF USE

(71) Applicant: Merit Medical Systems, Inc., South Jordan, UT (US)

(72) Inventors: David Craig Chadwick, Kearns, UT (US); Brian Stevens, Pleasant Grove, UT (US); William Padilla, Sandy, UT (US)

(21) Appl. No.: 14/460,650

(22) Filed: Aug. 15, 2014

Related U.S. Application Data
(60) Provisional application No. 61/866,616, filed on Aug. 16, 2013.

(51) Int. Cl. A61M 25/10

(52) U.S. Cl. CPC A61M 25/10182; A61M 2205/19; A61B 2017/00477
USPC 604/97.02

(57) ABSTRACT

Devices used to pressurize, depressurize, or otherwise displace fluid are disclosed. The devices may be configured to displace fluid along a line in order to inflate or deflate a medical device, such as a balloon. The devices may be configured to withstand high pressures. One or more portions of the devices may be reinforced. Additionally, the angles at which various components interact may be configured for use with certain pressures.

### RELATED APPLICATIONS

This application claims priority to U.S. Provisional Application No. 61/866,616, filed on Aug. 16, 2013 and titled “High-Pressure Inflation Devices and Methods of Use,” which is hereby incorporated by reference in its entirety.

### TECHNICAL FIELD

The present disclosure relates generally to devices used to pressurize, depressurize, or otherwise displace fluid, particularly in medical devices. More specifically, the present disclosure relates to high-pressure devices used to pressurize, depressurize, or otherwise displace fluid along a line in order to inflate or deflate a medical device, such as a balloon.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an inflation device.

FIG. 2 is a cross sectional view of the inflation device of FIG. 1 taken through plane 2-2.

FIG. 3 is an exploded view of the inflation device of FIG. 1.

FIG. 4A is a side view of a portion of the plunger shaft and handle of the inflation device of FIG. 1.

FIG. 4B is a cross sectional view of a coupling member of the inflation device of FIG. 1 taken through plane 2-2.

FIG. 4C is a cross sectional view of a portion of the plunger shaft and handle of the inflation device of FIG. 1 taken through plane 2-2.

FIG. 5A is a cross sectional view of the threaded portion of the inflation device of FIG. 1 in a first position.

FIG. 5B is a cross sectional view of the threaded portion of the inflation device of FIG. 5A in a second position.

FIG. 6 is a perspective view of the inflation device of FIG. 1 with fluid disposed within the inflation device and a balloon coupled to the inflation device.

### DETAILED DESCRIPTION

An inflation device may include a syringe which utilizes threads to advance or retract a plunger by rotating the plunger handle relative to the body of the syringe such that the threads cause longitudinal displacement of the plunger relative to the body. In some instances, an inflation syringe may further include retractable threads, enabling a practitioner to disengage the threads and displace the plunger by simply pushing or pulling the plunger.

The inflation syringe may comprise a coupling member configured to constrain movement of the plunger within the syringe body. The coupling member may comprise threads configured to engage with the retractable threads. In some instances, normally circular polymeric coupling members may “ovalize,” or in other words, deform to an oval shape, due to forces caused by pressure acting on the plunger (and thereby acting on the coupling member) within the syringe body. Ovalization may limit the maximum pressure at which a syringe may be used. For example, when a coupling member ovalizes, the retractable threads may spontaneously disengage from the coupling member, allowing the plunger to rapidly retract from within the syringe body. This results in a rapid pressure drop within the syringe body. When a medical device is attached to the inflation device, this also results in a rapid, unexpected pressure drop in the medical device. In some instances, pressure within the syringe body exceeding about 30 atmospheres (ATM) may cause ovalization of one or more portions of an inflation syringe. Conventionally, inflation devices may be configured for use at less than 30 ATM due to the degree of ovalization at 30 ATM or higher pressures. Inflation devices configured to resist ovalization may be utilized at higher pressures than such devices. Inflation devices within the scope of this disclosure may be configured for use in connection with pressure exceeding 30 ATM.

An inflation device may be configured such that the coupling member comprises a fiber-reinforced polymeric material capable of withstanding ovalization at high pressure. Alternatively or additionally, an inflation device may be configured so that the force required to spontaneously disengage the retractable threads from the coupling member is increased.

It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

The phrases “connected to,” “coupled to,” and “operably coupled to” refer to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest the practitioner during use. As specifically applied to the syringe portion of an inflation device, the proximal end of the syringe refers to the end nearest the handle and the distal end refers to the opposite end, the end nearest the inlet/outlet port of the syringe. Thus, if at one or more points in a procedure a physician changes the orientation of a syringe, as used herein, the term “proximal end” always refers to the handle end of the syringe (even if the distal end is temporarily closer to the physician).

“Fluid” is used in its broadest sense, to refer to any fluid, including both liquids and gases as well as solutions, compounds, suspensions, etc., which generally behave as fluids.

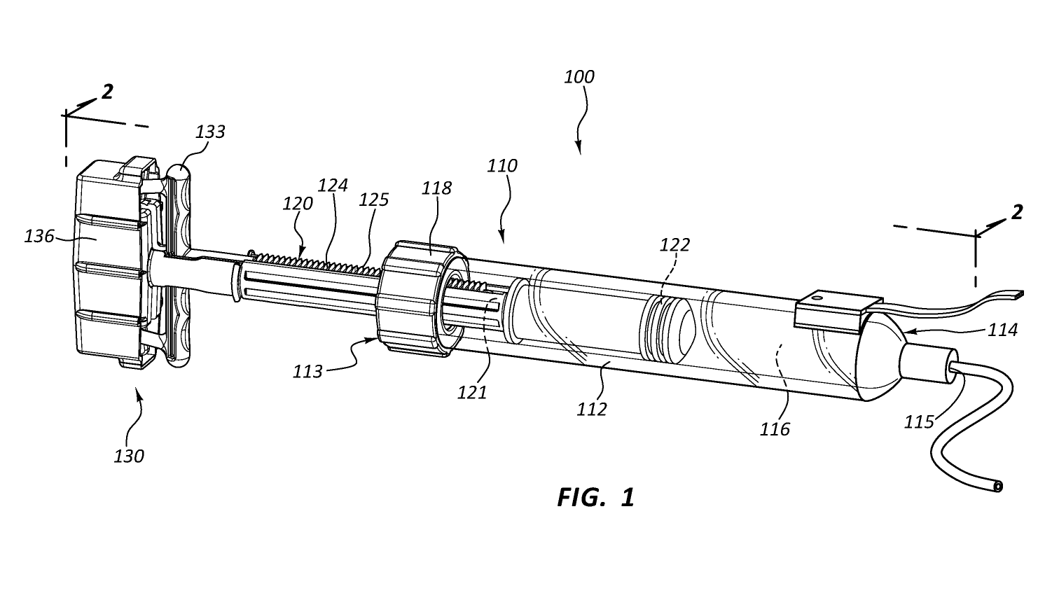

FIGS. 1-6 illustrate different views of an inflation device. In certain views the device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure.

FIG. 1 is a perspective view of an inflation device 100. In the illustrated embodiment, the inflation device 100 is partially comprised of a syringe 110. The inflation device 100 includes three broad groups of components; each group may have numerous subcomponents and parts. The three broad component groups are: a body component such as syringe body 112, a pressurization component such as plunger 120, and a handle 130.

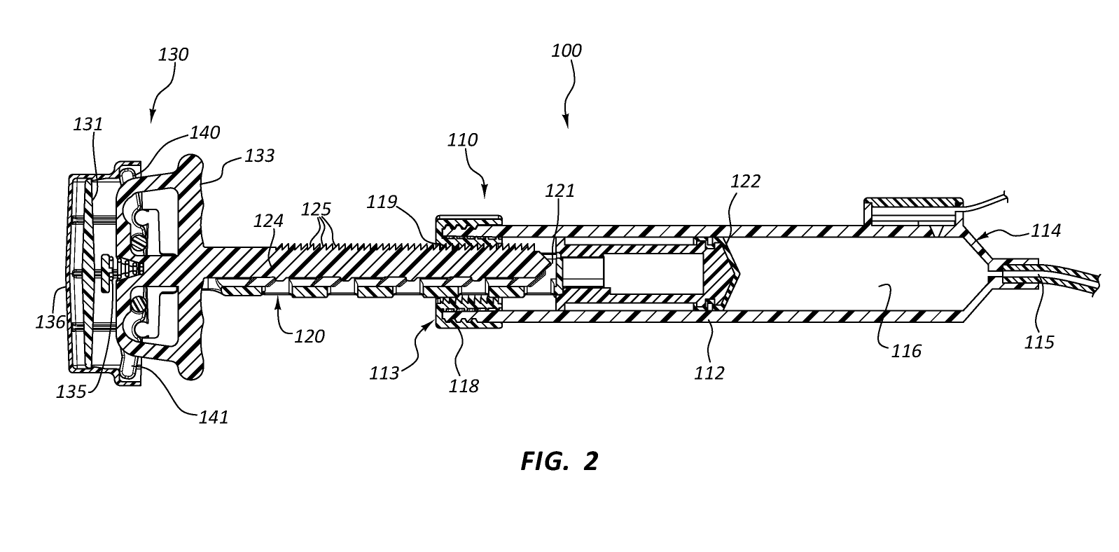

The syringe body 112 may be formed of a generally cylindrical hollow tube configured to receive the plunger 120. The syringe body 112 may include an inlet/outlet port 115 located adjacent the distal end 114 of the syringe body 112. In some embodiments, a coupling member 118 may be coupled to the syringe body 112 adjacent the proximal end 113 of the syringe body 112. The coupling member 118 may include a center hole configured to allow the plunger 120 to pass through the coupling member 118 into the syringe body 112. Further, the coupling member 118 may include coupling member threads 119 (FIG. 2) configured to selectively couple the coupling member 118 to the plunger 120.

The plunger 120 may be configured to be longitudinally displaceable within the syringe body 112. The plunger 120 may be comprised of a plunger shaft 121 coupled to a plunger seal 122 at the distal end of the plunger shaft 121. The plunger shaft 121 may also be coupled to the handle 130 at the proximal end of the plunger shaft 121, with the plunger shaft 121 spanning the distance between the plunger seal 122 and the handle 130.

The handle 130 broadly refers to the group of components coupled to the proximal end of the plunger 120, some of which may be configured to be graspable by a user. In certain embodiments, the handle 130 may be configured such that the user may manipulate the position of the plunger 120 by manipulating the handle 130. Further, in some embodiments, the handle 130 may be an actuator mechanism configured to manipulate components of the inflation device 100.

Any and every component disclosed in connection with any of the exemplary handle configurations herein may be optional. That is, though the handle 130 broadly refers to the components coupled to the proximal end of the plunger shaft 121 which may be configured to be graspable by a user, use of the term “handle” is not meant to indicate that every disclosed handle component is always present. Rather, the term is used broadly, referring to the collection of components, but not specifically referring to or requiring the inclusion of any particular component. Likewise, other broad groupings of components disclosed herein, such as the syringe 110 or syringe body 112 and the plunger 120, may also refer to collections of individual subcomponents. Use of these terms should also be considered non-limiting, as each subcomponent may or may not be present in every embodiment.

As shown in FIG. 1, a fluid reservoir 116 may be defined by the space enclosed by the inside walls of the syringe body 112 between the plunger seal 122 and the distal end 114 of the syringe body 112. Accordingly, movement of the plunger seal 122 with respect to the syringe body 112 will alter the size and volume of the fluid reservoir 116.

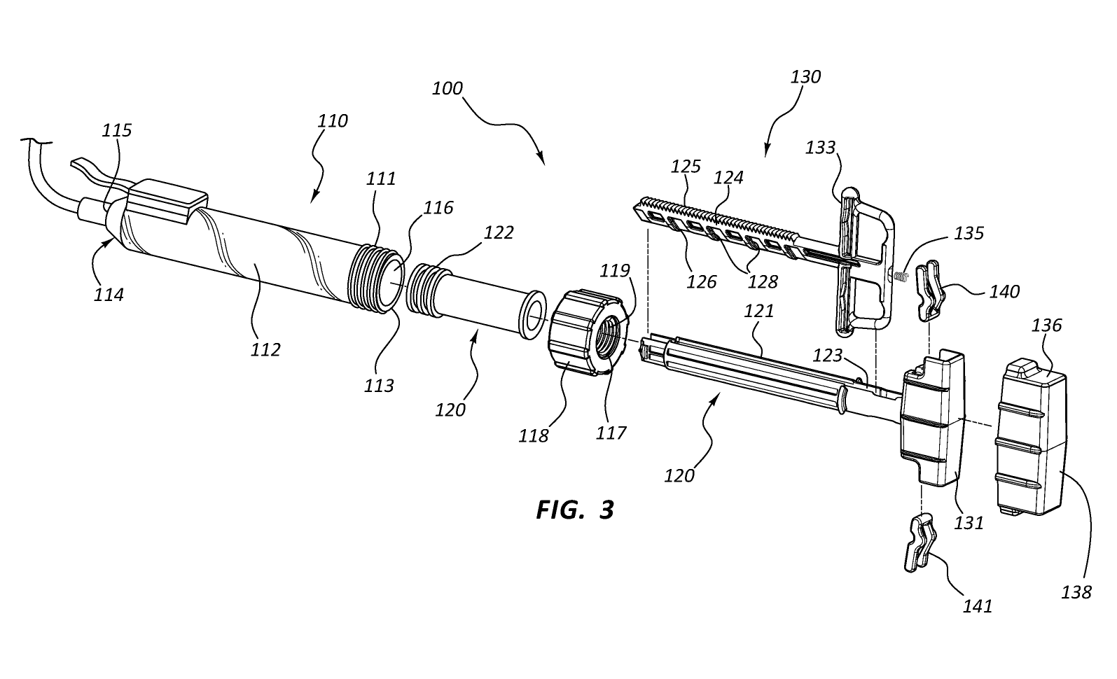

As shown in FIGS. 1 and 2, in some embodiments, the syringe 110 may include a coupling member 118, fixedly coupled to the proximal end 113 of the syringe body 112. The coupling member 118 may utilize threads 117 (FIG. 3) or other coupling mechanisms to fixedly couple the coupling member 118 to corresponding threads 111 on the syringe body 112. The coupling member 118 may additionally include coupling member threads 119 configured to couple the coupling member 118 to a portion of the plunger 120. The plunger 120 may also include external plunger threads 125 configured to couple the plunger 120 to the coupling member 118. The plunger 120 may thus be translated longitudinally with respect to the syringe body 112 by rotating the plunger 120 such that the interaction of the coupling member threads 119 and the plunger threads 125 results in the longitudinal translation of the plunger 120. Thus, when the plunger threads 125 and the coupling member threads 119 are engaged, movement of the plunger 120 is constrained with respect to the syringe body 112, though the plunger 120 is not necessarily fixed with respect to the syringe body 112. For example, the plunger 120 may be rotatable, but not directly translatable, when the threads 125, 119 are engaged.

The plunger threads 125 may be configured such that they may be retracted within the plunger shaft 121. As shown in FIG. 3, in some embodiments, the plunger threads 125 do not extend 360 degrees around the axis of the plunger shaft 121. Furthermore, as shown in FIGS. 3-5B, the plunger threads 125 may be formed on a thread rail 124, which may be disposed within a groove 123 in the plunger shaft 121.

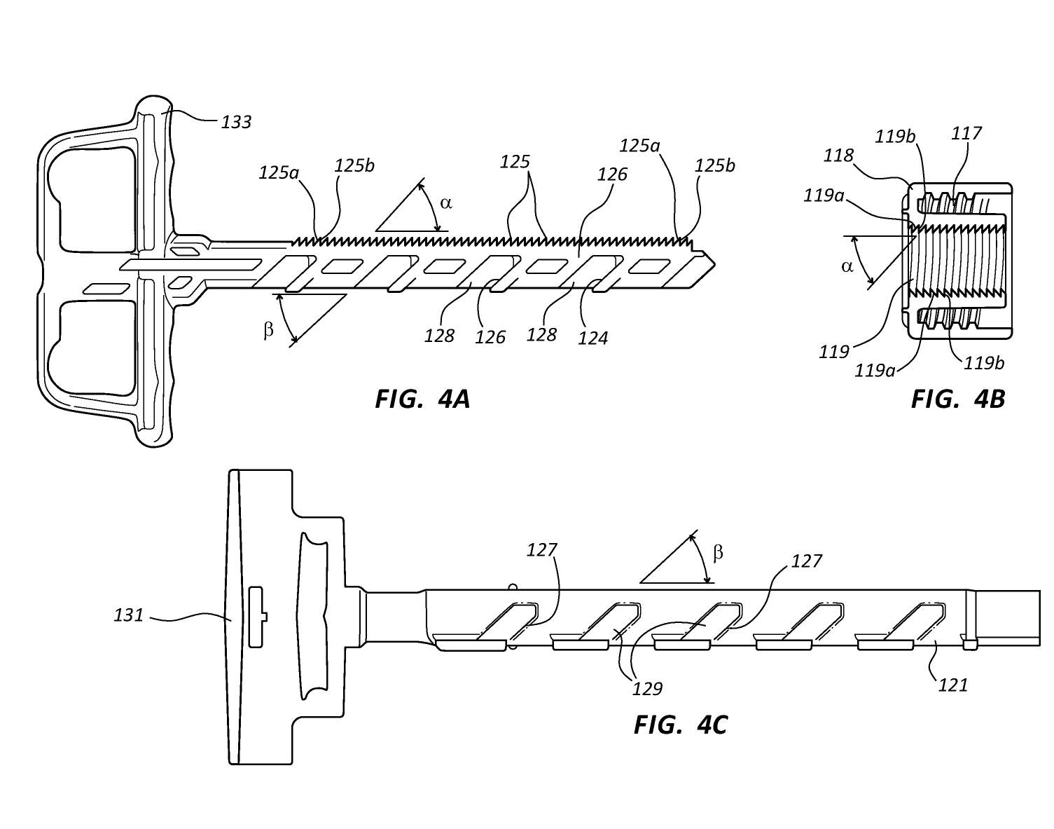

FIG. 4A illustrates a side view of the thread rail 124 and a component of the handle 130, trigger 133. FIG. 4B illustrates a cross sectional view of the coupling member 118 taken through plane 2-2. FIG. 4C illustrates a cross sectional view of the interior of the groove 123 of the plunger shaft 121 taken through plane 2-2. FIG. 4C also illustrates an inner member 131 of the handle 130. The components of the handle 130 in the illustrated embodiment are discussed in more detail below.

The plunger threads 125 may be configured to engage with the coupling member threads 119 at an angle alpha (α). “Engage,” as used in this context, refers to the final angle at which the plunger threads 125 fully mesh with the coupling member threads 119. In the illustrated embodiment, this final angle is primarily governed by the angle of the proximal surface 125a of the ridges of the plunger threads 125 and the distal surface 119a of the ridges of the coupling member threads 119 (viewing each of the thread ridges as a separate ridge, even though in the illustrated embodiment, the coupling member threads 119 are formed by one continuous spiraling ridge). In the illustrated embodiment, the distal surface 125b of the ridges of the plunger threads 125 and the distal surface 119b of the ridges of the coupling member threads 119 are essentially perpendicular to the longitudinal axis of the inflation device 100.

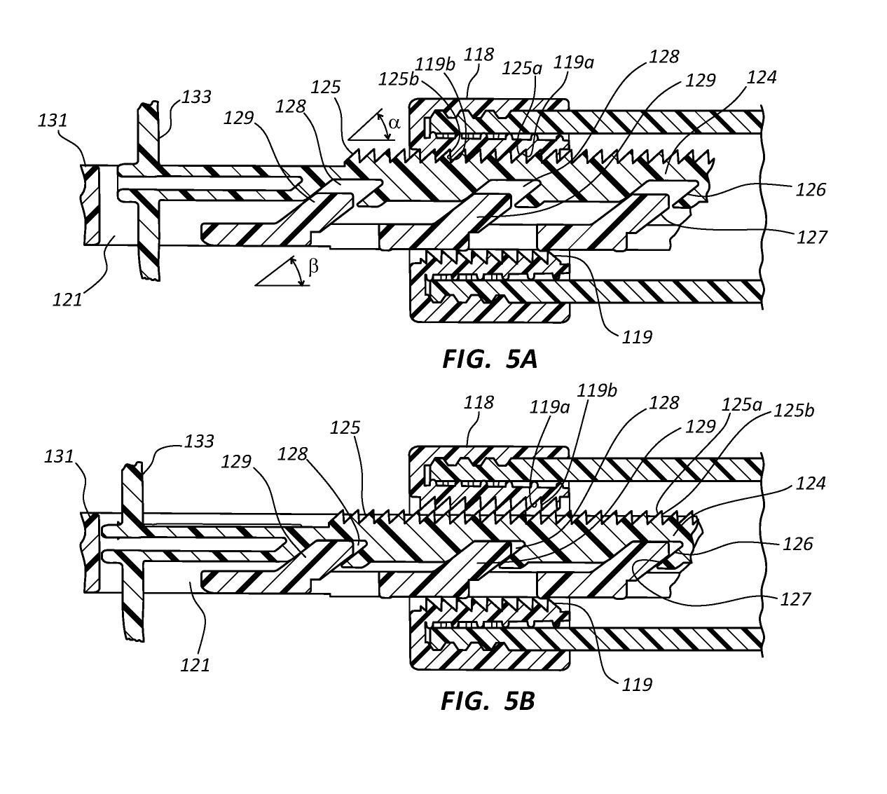

The thread rail 124 may be configured with angled surfaces 126 on the sides of slots 128. The groove 123 may be configured with angled surfaces 127 on the sides of protrusions 129. The protrusions 129 extend inwardly and symmetrically from opposing inner sides of the groove 123 (FIG. 4C). The slots 128 and the protrusions 129, via angled surfaces 126 and 127, are configured to interact at an angle beta (β), such that the plunger threads 125 may be retractable within the plunger shaft 121 at the angle beta. The relationship between the slots 128 and the protrusions 129 within the groove 123 (FIG. 3) is shown in FIGS. 4A, 4B, 5A, and 5B.

A net force applied to the thread rail 124 in the proximal direction may thus simultaneously cause the thread rail 124 to translate in the proximal direction and to retract toward the center axis of the plunger shaft 121 due to the interaction of the angled surfaces 126 on the slots 128 with the angled surfaces 127 of the protrusions 129. Similarly, a net force applied to the thread rail 124 in the distal direction may cause the thread rail 124 to translate in the distal direction and to move away from the center axis of the plunger shaft 121 and toward threads 119 of the coupling member 118. Thus, a net force applied to the thread rail 124 in the proximal or distal direction is divided into a proximal or distal component and a radially inward or outward component due to the interaction of the angled surfaces 126 on the slots 128 and the angled surfaces 127 on the protrusions 129. Changing the angle beta may thus change the percentage of the net force which is translated into the proximal/distal and radially outward/inward forces.

In the illustrated embodiment, a distally oriented biasing force acting on the thread rail 124 may bias the plunger threads 125 to the engaged position. It will be appreciated by one of ordinary skill in the art having the benefit of this disclosure that it is within the scope of this disclosure to modify the angles and interfaces such that a distally oriented biasing force on the thread rail 124 would bias the plunger threads 125 in the retracted position. Analogous mechanisms are disclosed in U.S. Pat. Nos. 5,047,015; 5,057,078; 5,163,904; and 5,209,732, which are each incorporated by reference in their entireties.

FIGS. 5A and 5B illustrate two possible positions of the thread rail 124 with respect to the coupling member threads 119 and the plunger shaft 121. FIG. 5A shows thread rail 124 disposed in an engaged position, such that the plunger threads 125 are engaged with the coupling member threads 119. FIG. 5B shows the thread rail 124 sufficiently retracted into the plunger shaft 121 that the plunger threads 125 are not engaged with the coupling member threads 119.

The plunger threads 125 may be configured to be retracted from the coupling member threads 119 at the angle beta that is different from the angle alpha. The angle alpha may be greater than the angle beta. The angle alpha may be sufficiently greater than the angle beta that the inflation device 100 is capable of withstanding pressures within reservoir 116 that exceed 30 atmospheres (ATM). However, the angle alpha and the angle beta may be similar enough that distal pressure on the thread rail 124 is capable of causing full engagement between the coupling member threads 119 and the plunger threads 125. For example, the angle alpha may be greater than the angle beta by about 2 degrees to about 10 degrees, including by about 3 degrees, by about 4 degrees, by about 5 degrees, by about 6 degrees, by about 7 degrees, by about 8 degrees, and by about 9 degrees. In particular, the angle alpha may be greater than the angle beta by about 3 degrees to about 7 degrees. In the illustrated embodiment, the angle alpha is greater than the angle beta by about 5 degrees.

With the plunger threads 125 engaged with the coupling member threads 119, as pressure builds in the reservoir 116, the forces acting on the proximal surfaces 125a and the distal surfaces 119a increase in the distal longitudinal direction. These forces may translate into a force at the angle alpha that may tend to push the thread rail 124 and the plunger threads 125 away from engagement with the coupling member threads 119. When the angle alpha is the same as the angle beta, the forces acting on the proximal surfaces 125a and the distal surfaces 119a and the forces acting on the angled surfaces 126 and 127 may be about the same. When the angle alpha is greater than the angle beta, the forces acting on the angled surfaces 126 and 127 at the angle beta are decreased, relative to the forces acting on the proximal surfaces 125a and the distal surfaces 119a at the angle alpha. The forces acting at the angle beta as pressure builds within the reservoir 116 may partially govern at what pressure the thread rail 124 spontaneously retracts from the coupling member 118. Accordingly, the forces acting at the angle beta may partially determine the maximum pressure at which the reservoir 116 may be pressurized prior to failure of the inflation device 100.

Accordingly, comparing two inflation devices with coupling members made of the same material, but where a first one has an alpha angle greater than the beta angle, such as in the illustrated embodiment, and a second one has an alpha angle equal to the beta angle, the first inflation device may be able to withstand greater pressures than the second inflation device. Thus, the force required to spontaneously disengage the plunger threads 125 from the coupling member 118 may be increased in the first inflation device (i.e., the inflation device 100). Additionally, increasing the force required to spontaneously disengage the plunger threads 125 from the coupling member 118 by changing angles alpha and beta may also increase the net force applied to the thread rail 124 by a practitioner to disengage plunger threads 125. Conventionally, these angles would not be altered due to this change in disengagement force.

As an alternative to having the alpha angle greater than the beta angle, the coupling member 118 may be comprised of a material with greater structural stiffness than the material of the plunger shaft 121. For example, the inflation device 100 may be configured with an alpha angle that is the same as the beta angle, but has a coupling member 118 made of a fiber-reinforced polymeric material. The material may comprise a fiberglass-reinforced plastic, such as fiberglass-reinforced nylon, or alternatively, a polymer reinforced with carbon, basalt or aramid fibers. However, the coupling member 118 may be made of any material, polymeric or otherwise, capable of resisting ovalization at the desired maximum pressures for the inflation device 100. Inflation devices comprising a coupling member comprising a fiber-reinforced polymeric material may be more capable of resisting ovalization compared to inflation devices comprising a coupling member made of the same material as the plunger shaft.

In some embodiments, the inflation device 100 is comprised of sterilization compatible materials. “Sterilization compatible materials,” as used herein, refers to materials capable of being sterilized without rendering the materials unsuitable for their intended purpose. If a material is configured for sterilization by at least one method of sterilization without being rendered unsuitable for its intended purpose, then the material is a “sterilization compatible material.” For example, a polymeric coupling member 118 may deform when autoclaved at temperatures sufficient to sterilize the barrel, rendering the barrel unsuitable for its intended purpose of maintaining a seal with a circular plunger. However, if the same polymeric coupling member 118 is sterilized by another sterilization technique, such as irradiation, and maintain suitability for its intended purpose, then the polymeric material is a “sterilization compatible material.”

In some embodiments, the inflation device 100 is comprised of irradiation compatible materials. “Irradiation compatible materials,” as used herein, refers specifically to materials capable of being sterilized by irradiation without rendering the materials unsuitable for their intended purpose. For example, the coupling member 118 may comprise a material that upon irradiation changes in physical properties such that the syringe is unsuitable for its intended purpose. For example, irradiation may alter certain surface properties of the coupling member threads 119 such that unacceptably high friction would result from attempts to rotate the plunger threads 125 while engaged with the coupling member threads 119.

In some embodiments where the alpha angle is greater than the beta angle, such as in the illustrated embodiment, the coupling member may also be comprised of a fiber-reinforced polymeric material.

The inflation device 100 may be configured to withstand reservoir 116 pressures that exceed about 30 atmospheres (ATM). The inflation device 100 may be configured to withstand reservoir 116 pressures that exceed about 35 ATM. The inflation device 100 may be configured to withstand reservoir 116 pressures that exceed about 40 ATM. The inflation device 100 may be configured to withstand reservoir 116 pressures that exceed about 45 ATM. The inflation device 100 may be configured to withstand reservoir 116 pressures that exceed about 50 ATM. At the above pressures, the coupling member 118 may be configured to resist spontaneous disengagement from the plunger threads 125 and/or the coupling member 118 may be configured to resist ovalization. The pressure capabilities of the inflation device 100 may arise in part from the alpha angle being greater than the beta angle, from the coupling member 118 comprising a fiber-reinforced polymeric material, or both.

The retractable threads may allow a user to displace the plunger shaft 121 relative to the syringe body 112 either through rotation of the plunger shaft 121 (and the subsequent interaction of threads), or by retracting the plunger threads 125 and displacing the plunger shaft 121 by applying opposing forces on the plunger shaft 121 and the syringe body 112. (The forces, of course, may move the plunger shaft 121 distally or proximally with respect to the syringe body 112.) Both methods of displacement may be utilized during the course of a single therapy.

In some instances, a practitioner may desire to quickly displace the plunger shaft 121, for instance, while priming the inflation device or while priming or deflating an attached medical device such as a balloon. Quick displacement of the plunger shaft 121 may be accomplished by retracting the plunger threads 125 and sliding the plunger shaft 121 relative to the syringe body 112. For example, a practitioner may quickly fill the reservoir 116 with fluid by disengaging the plunger threads 125 and pulling the plunger shaft 121 in a proximal direction with respect to the syringe body 112. Further, a practitioner may quickly force fluid into lines leading to a medical device or quickly expel unwanted air bubbles from the reservoir 116 by retracting the plunger threads 125 and repositioning the plunger shaft 121.

In other instances, the practitioner may desire more precise control over the position of the plunger shaft 121 (for example when displacing the plunger shaft 121 in order to adjust the fluid pressure within the reservoir 116) or it may simply be difficult or impossible without a mechanical advantage to displace the plunger shaft 121 due to high fluid pressure within the reservoir 116. In these instances, the practitioner may opt to displace the plunger shaft 121 by rotation of the plunger shaft 121.

Similar principles of operation of the inflation device 100 may be achieved with different configurations of the inflation device. For example, the coupling member 118 may be integrally formed with the syringe body 112. In that embodiment threads 111 and threads 117 may not be present. In another example, the coupling member 118 may be rotatably coupled to the syringe body 112, such as via a rotatable hub. In such embodiments, rotation of the coupling member 118 may insert or retract the plunger 120 within the syringe body 112 when the plunger 120 is engaged with the coupling member 118. For example, the coupling member 118 may be rotated counter-clockwise while the plunger shaft 121 is rotated clockwise to advance the plunger 120. The coupling member 118 may comprise additional features, such as levers, to facilitate mechanical advantage in the rotation of the coupling member 118.

In the illustrated embodiment, the inflation device 100 is configured to provide a mechanical advantage when engaging or disengaging the coupling member 118. Referring back to FIG. 3, the handle 130 of the inflation device 100 may include components which enable a practitioner to retract the thread rail 124 of the plunger 120. In some embodiments, the plunger shaft 121 may be fixed to a first member such as inner member 131 of the handle 130. The thread rail 124 may be fixed to a trigger 133 component of the handle. Further, a biasing component 135 may be configured to bias the trigger 133 in a distal direction. Because the trigger 133 is fixed to the thread rail 124, a distally oriented force on the trigger 133 will result in a distally oriented force on the thread rail 124 as well. The force provided by the biasing component 135 (hereafter referred to as the biasing force) may thus bias the thread rail 124 in the engaged position as described above. Conversely, overcoming the biasing force and translating the trigger 133 in a proximal direction with respect to the plunger shaft 121 and inner member 131 may retract the plunger threads 125.

In some embodiments the handle 130 may further include a second member such as outer sleeve 136 and one or more levers 140, 141. The levers 140, 141 may be disposed such that they provide mechanical advantage, enabling the user to more easily overcome the biasing force and draw the trigger 133 toward the inner member 131. Any configuration for providing mechanical advantage in operation of an inflation device, such as the configurations disclosed in U.S. Patent Publication No. 2013-0123693, the contents of which are incorporated herein by reference in their entirety, may be used with the inflation devices disclosed herein, with the aid of the present disclosure.

A handle configured to provide a mechanical advantage when retracting a thread rail may be desirable for certain therapies which require large syringes or high pressure. Such therapies may also require a larger biasing force due to the size of the device or the pressure within the device. A handle providing a mechanical advantage may make devices configured for such therapies easier to use.

In some embodiments, the handle 130 is not configured to provide a mechanical advantage when disengaging the coupling member 118. For example, the levers 140 and 141 may not be present. In such embodiments, a user may need to directly overcome the biasing force of the biasing component 135 to disengage the plunger threads 125 of the thread rail 124 from the coupling member threads 119.

Many design modifications relating to the outer sleeve 136 are within the scope of the current disclosure. For example, in the illustrated embodiments, the outer sleeve 136 has a cap-like shape, fitting over the inner member 131. In other embodiments, the outer sleeve 136 may instead be designed as a button which slides into the inner member 131 when it is compressed. Likewise, any other longitudinally actuatable component may be utilized in place of the outer sleeve 136.

The handle mechanism described above, and shown in each of FIGS. 2-5B, may also be utilized to change the location and direction of an input force required to retract the plunger threads 125. Essentially, the mechanism allows a user to draw the trigger 133 toward the inner member 131 (and thus retract the threads) solely by applying a distally oriented force to the top surface 138 of the outer sleeve 136. As outlined above, the levers 140, 141 transfer this force to the trigger 133, which retracts the plunger threads 125.

In some instances a user, such as a medical practitioner, may desire to displace the plunger 120 in a distal direction with only one hand. This may be accomplished by grasping the syringe body 112 and using a surface, for example a table top, to apply a distally oriented force on the top surface 138 of the outer sleeve 136. In this manner, a mechanism such as that described above may enable a practitioner to displace the plunger in a one-handed fashion.

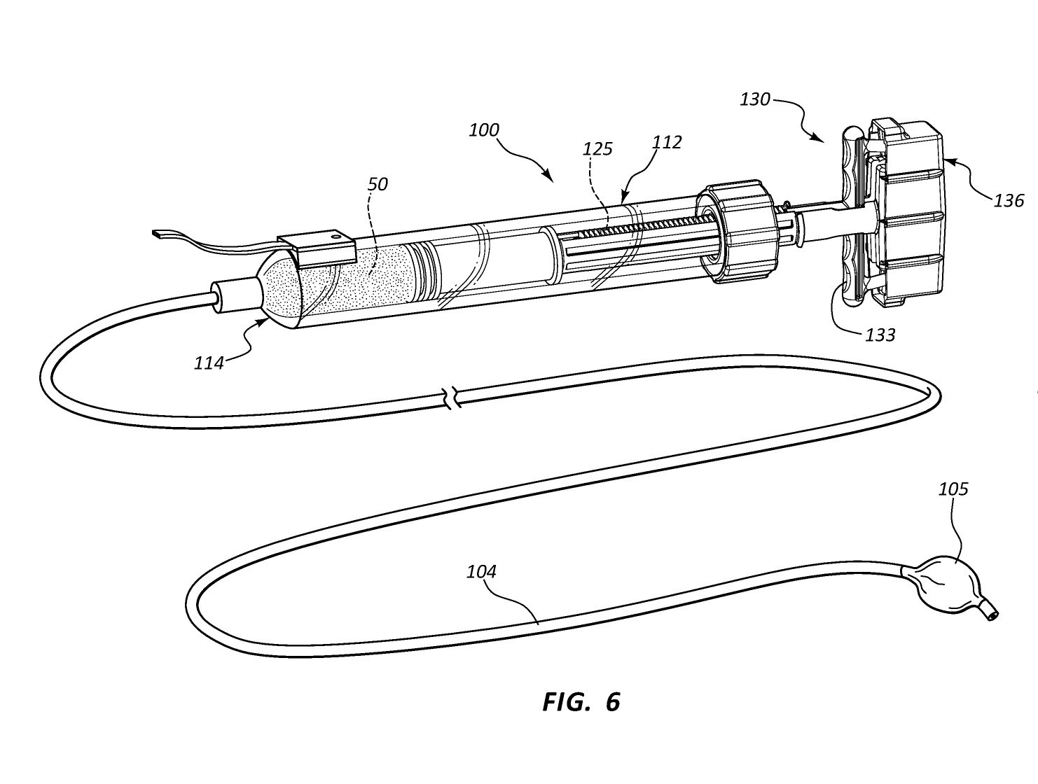

FIG. 6 is a perspective view of the inflation device 100 with fluid 50 disposed within the device and a balloon 105 coupled to the inflation device 100 via a delivery line 104. Referring now to components shown in FIG. 6 as well as the other figures, in some instances it may be desirable to operate the syringe 110 “one-handed” as described above in order to prime the system. For example, a practitioner may utilize the inflation device 100 in connection with a therapy which includes the balloon 105, such as an angioplasty. The practitioner may initially fill the syringe body 112 with fluid 50, such as a contrast fluid, by drawing the plunger 120 back in the proximal direction. In some instances, the practitioner will do so by grasping the handle 130 of the inflation device 100 with a first hand, while grasping the syringe body 112 with a second hand. The practitioner may then retract the plunger threads 125 by squeezing the trigger 133 and the outer sleeve 136 together with his or her first hand, then drawing the plunger 120 back in the proximal direction.

After a desired amount of fluid is disposed within the syringe body 112, the practitioner may orient the syringe such that the distal end 114 of the syringe body 112 is above the handle 130, so any air bubbles in the fluid will tend to rise to the distal end 114 of the syringe body 112. The practitioner may also shake, tap, or otherwise disturb the syringe 110 in order to facilitate movement of any air bubbles in the fluid. The practitioner may then prime the syringe 110 by displacing the plunger 120 in a distal direction with respect to the syringe body 112, thereby forcing the air bubbles from the syringe body 112.

In some instances the practitioner will displace the plunger 120 as described after first retracting the plunger threads 125. This may be accomplished in any manner disclosed herein, including the one-handed operation described above. That is, the practitioner may prime the inflation device simply by grasping the syringe body 112 with one hand and using a fixed object or surface, such as a table top, to exert a distally directed force on the top surface 138 of the outer sleeve 136. The force on the outer sleeve 136 will both (1) retract the plunger threads 125 via the handle 130 mechanism and (2) act to displace the plunger 120 in a distal direction with respect to the syringe body 112. This orientation positions the syringe body 112 in a potentially desirable position to allow air to travel to the distal end 114 of the syringe body 112 while simultaneously orienting the handle 130 such that the top surface 138 of the outer sleeve 136 directly faces a horizontal surface such as a table. Thus, in some instances a practitioner may desire to prime the syringe 110 in this way due to the orientation of the syringe 110 as well as the ability to do so with one hand.

### Exemplary Embodiments

The following embodiments are illustrative and exemplary and not meant as a limitation of the scope of the present disclosure in any way.

I. Reinforced Inflation Devices

In one embodiment, an inflation device configured for use in connection with a medical device may comprise: (1) a body component; (2) a pressurization component configured to increase or decrease pressure within the body component by displacing the pressurization component with respect to the body component; (3) a coupling member configured to selectively constrain the displacement of the pressurization component with respect to the body component, wherein the coupling mechanism comprises a fiber-reinforced polymeric material; and (4) an actuator configured to selectively engage and disengage the coupling member.

The fiber-reinforced polymeric material may comprise a fiberglass-reinforced plastic.

The plastic may comprise a nylon.

The coupling mechanism may be configured to resist ovalization when pressure within the body component exceeds about 30 atmospheres (ATM).

The coupling mechanism may be configured to resist ovalization when pressure within the body component exceeds about 35 atmospheres (ATM).

The coupling mechanism may be configured to resist ovalization when pressure within the body component exceeds about 40 atmospheres (ATM).

The coupling mechanism may be configured to resist ovalization when pressure within the body component exceeds about 45 atmospheres (ATM).

The coupling mechanism may be configured to resist ovalization when pressure within the body component exceeds about 50 atmospheres (ATM).

The actuator may be configured to provide a mechanical advantage in engaging or disengaging the coupling mechanism.

The actuator may be configured to disengage the coupling mechanism in response to a proximally oriented force on the actuator.

The body component may comprise a syringe body.

The pressurization component may comprise a plunger configured to form a slidable seal with an internal surface of the syringe body and configured for insertion and retraction within the syringe body.

The coupling member may comprise coupling member threads configured to constrain movement of the plunger within the syringe body and wherein the plunger is configured to selectively engage and disengage with the coupling member threads.

The coupling member may be coupled to the syringe body.

The coupling member may be integrally molded with the syringe body.

The coupling member may be rotatably coupled to the syringe body, wherein rotation of the coupling member inserts or retracts the plunger within the syringe body when the plunger is engaged with the coupling member.

The coupling member may be fixedly coupled to the syringe body, wherein rotation of the plunger inserts or retracts the plunger within the syringe body when the plunger is engaged with the coupling member.

The coupling member may comprise a central hole configured to allow a portion of the plunger to pass through the hole.

The coupling member threads may be formed on the internal surface of the central hole.

A shaft of the plunger may comprise plunger threads configured to be selectively engaged with the coupling member threads.

The plunger shaft may be configured to screw into or out of the syringe body when the plunger threads are engaged with the coupling member threads.

The plunger shaft may be configured to slide into or out of the syringe body when the plunger threads are disengaged from the coupling member threads.

The plunger threads may be configured for retraction from or advancement to the coupling member threads.

The plunger threads may be configured to engage the coupling member threads at a first angle.

The plunger threads may be formed on a thread rail configured for retraction from or advancement to the surface of the plunger shaft.

The thread rail may be configured to be retracted from the coupling member threads at a second angle.

The first angle may be the same as the second angle.

The first angle may be greater than the second angle.

The actuator may be operably coupled to the thread rail and configured to selectively retract and advance the thread rail.

The inflation device may comprise a handle operably coupled to the plunger shaft and to the actuator.

The handle may be configured to provide a mechanical advantage when retracting the thread rail with the actuator.

The handle may comprise a lever operably connected to the actuator, wherein the lever is configured to provide a mechanical advantage when retracting the thread rail with the actuator.

The coupling member threads may be configured to resist spontaneous disengagement from the plunger when pressure acting on the plunger within the syringe body exceeds about 30 atmospheres (ATM).

The coupling member threads may be configured to resist spontaneous disengagement from the plunger when pressure acting on the plunger within the syringe body exceeds about 35 atmospheres (ATM).

The coupling member threads may be configured to resist spontaneous disengagement from the plunger when pressure acting on the plunger within the syringe body exceeds about 40 atmospheres (ATM).

The coupling member threads may be configured to resist spontaneous disengagement from the plunger when pressure acting on the plunger within the syringe body exceeds about 45 atmospheres (ATM).

The coupling member threads may be configured to resist spontaneous disengagement from the plunger when pressure acting on the plunger within the syringe body exceeds about 50 atmospheres (ATM).

The inflation device may be configured for inflation of a medical device.

The inflation device may be comprised of sterilization compatible materials.

The inflation device may be comprised of irradiation compatible materials.

II. Angles of Inflation Devices

In one embodiment, an inflation device comprises: (1) a syringe body; (2) a plunger configured to form a slidable seal with an internal surface of the syringe body and configured for insertion and retraction within the syringe body; and (3) a coupling member comprising coupling member threads configured to constrain movement of the plunger within the syringe body; and the plunger may comprise plunger threads configured to be selectively engaged and disengaged with the coupling member threads, wherein the plunger threads are configured to be retractable from the coupling member threads; and the plunger threads may be configured to engage with the coupling member threads at a first angle and wherein the plunger threads are configured to be retracted from the coupling member threads at a second angle that is different from the first angle.

The first angle may be greater than the second angle.

The first angle may be greater than the second angle by about 2 degrees to about 10 degrees.

The first angle may be greater than the second angle by about 3 degrees to about 7 degrees.

The coupling member may be coupled to the syringe body.

The coupling member may be integrally molded with the syringe body.

The coupling member may be rotatably coupled to the syringe body, and rotation of the coupling member may insert or retract the plunger within the syringe body when the plunger is engaged with the coupling member.

The coupling member may be fixedly coupled to the syringe body, and rotation of the plunger may insert or retract the plunger within the syringe body when the plunger is engaged with the coupling member.

The coupling member may comprise a central hole configured to allow a portion of the plunger to pass through the hole.

The coupling member threads may be formed on the internal surface of the central hole.

A shaft of the plunger may comprise the plunger threads configured to be selectively engaged with the coupling member threads.

The plunger shaft may be configured to screw into or out of the syringe body when the plunger threads are engaged with the coupling member threads.

The plunger shaft may be configured to slide into or out of the syringe body when the plunger threads are disengaged from the coupling member threads.

The plunger threads may be formed on a thread rail configured for retraction from or advancement to the surface of the plunger shaft.

The thread rail may be configured to be retracted from the coupling member threads at the second angle.

The inflation device may comprise an actuator operably coupled to the thread rail and configured to selectively retract and advance the thread rail.

The inflation device may comprise a handle operably coupled to the plunger shaft and to the actuator.

The handle may be configured to provide a mechanical advantage when retracting the thread rail with the actuator.

The handle may comprise a lever operably connected to the actuator, and the lever may be configured to provide a mechanical advantage when retracting the thread rail with the actuator.

The coupling member may comprise a fiber-reinforced polymeric material.

The fiber-reinforced polymeric material may comprise a fiberglass-reinforced material.

The fiber-reinforced polymeric material may comprise a fiberglass-reinforced nylon material.

The coupling member threads may be configured to resist spontaneous disengagement from the plunger when pressure acting on the plunger within the syringe body exceeds about 30 atmospheres (ATM).

The coupling member threads may be configured to resist spontaneous disengagement from the plunger when pressure acting on the plunger within the syringe body exceeds about 35 atmospheres (ATM).

The coupling member threads may be configured to resist spontaneous disengagement from the plunger when pressure acting on the plunger within the syringe body exceeds about 40 atmospheres (ATM).

The coupling member threads may be configured to resist spontaneous disengagement from the plunger when pressure acting on the plunger within the syringe body exceeds about 45 atmospheres (ATM).

The coupling member threads may be configured to resist spontaneous disengagement from the plunger when pressure acting on the plunger within the syringe body exceeds about 50 atmospheres (ATM).

The inflation device may be configured for inflation of a medical device.

The inflation device may be comprised of sterilization compatible materials.

The inflation device may be comprised of irradiation compatible materials.

III. Methods of Pressurization

In one embodiment, a method of pressurizing a medical device comprises: (1) obtaining an inflation device comprising a syringe body, a plunger within the syringe body, and a handle coupled to the plunger; (2) decoupling the plunger from the syringe body; and (3) translating the plunger within the syringe body sufficient to generate pressures exceeding 30 atmospheres (ATM) within the syringe body.

The inflation device may comprise a handle configured to selectively couple and decouple the plunger from the syringe body.

The syringe body may be configured to couple and decouple with the plunger comprising a fiber-reinforced polymeric material.

Translating the plunger within the syringe body sufficient to generate pressures exceeding 30 atmospheres within the syringe body may comprise translating the plunger a first axial distance with the plunger decoupled from the syringe body to generate a first pressure.

The method may comprise coupling the plunger to the syringe body such that threads on the plunger engage with threads on the syringe body.

The method may comprise rotating the plunger to translate the plunger a second axial distance to generate a second pressure.

The second axial distance may be distally greater than the first axial distance and the second pressure is higher than the first pressure.

The second axial distance may be distally less than the first axial distance and the second pressure is lower than the first pressure.

The threads on the plunger may be configured to engage and disengage with the threads on the syringe body at a first angle, and the plunger may be configured to couple and decouple from the syringe body at a second angle, and the first angle may be greater than the second angle.

Without further elaboration, it is believed that one skilled in the art can use the preceding description to utilize the present disclosure to its fullest extent. The examples and embodiments disclosed herein are to be construed as merely illustrative and exemplary, and not as a limitation of the scope of the present disclosure in any way. It will be apparent to those having skill in the art that changes may be made to the details of the above-described embodiments without departing from the underlying principles of the disclosure herein. It is intended that the scope of the invention be informed by the claims appended hereto and their equivalents.

1. An inflation device comprising:
a syringe body;
a plunger configured to form a slidable seal with an internal surface of the syringe body and configured for insertion and retraction within the syringe body; and
a coupling member comprising coupling member threads configured to constrain movement of the plunger within the syringe body;
wherein the plunger comprises plunger threads configured to be selectively engaged and disengaged with the coupling member threads, wherein the plunger threads are configured to be retractable from the coupling member threads;
wherein the plunger threads are configured to engage with the coupling member threads at a first angle and wherein the plunger threads are configured to be retracted from the coupling member threads at a second angle that is different from the first angle.

2. The inflation device of claim 1, wherein the first angle is greater than the second angle.

3. The inflation device of claim 1, wherein the first angle is greater than the second angle by about 2 degrees to about 10 degrees.

4. The inflation device of claim 1, wherein the first angle is greater than the second angle by about 3 degrees to about 7 degrees.

5. The inflation device of claim 1, wherein the coupling member is integrally molded with the syringe body.

6. The inflation device of claim 1, further comprising an actuator operably coupled to the plunger and configured to couple the plunger to the coupling member.

7. The inflation device of claim 6, wherein the actuator is configured to provide a mechanical advantage.

8. The inflation device of claim 1, wherein the coupling member comprises a fiber-reinforced polymeric material.

9. The inflation device of claim 1, wherein the coupling member threads are configured to resist spontaneous disengagement from the plunger when pressure acting on the plunger within the syringe body exceeds about 30 atmospheres (ATM).

10. An inflation device configured for use in connection with a medical device, the inflation device comprising:
a body component;
a pressurization component configured to increase or decrease pressure within the body component by displacing the pressurization component with respect to the body component;
a coupling member configured to selectively constrain the displacement of the pressurization component with respect to the body component, wherein the coupling mechanism comprises a fiber-reinforced polymeric material; and
an actuator configured to selectively engage and disengage the coupling member.

11. The inflation device of claim 10, wherein the fiber-reinforced polymeric material comprises a fiberglass-reinforced plastic.

12. The inflation device of claim 10, wherein the coupling mechanism is configured to resist ovalization when pressure within the body component exceeds about 30 atmospheres (ATM).

13. The inflation device of claim 10, wherein the coupling mechanism is configured to resist ovalization when pressure within the body component exceeds about 50 atmospheres (ATM).

14. The inflation device of claim 10, wherein the actuator is configured to provide a mechanical advantage in engaging or disengaging the coupling mechanism.

15. The inflation device of claim 10, wherein the pressurization component comprises pressurization component threads configured for retraction from or advancement to the coupling member threads disposed on the coupling member.

16. The inflation device of claim 15, wherein the pressurization component threads are configured to engage the coupling member threads at a first angle.

17. The inflation device of claim 16, wherein the pressurization component is configured to be retracted from the coupling member threads at a second angle.

18. The inflation device of claim 17, wherein the first angle is the same as the second angle.

19. The inflation device of claim 17, wherein the first angle is greater than the second angle.

20. A method of pressurizing a medical device, comprising:
obtaining an inflation device comprising a syringe body, a plunger within the syringe body, and a handle coupled to the plunger;
decoupling the plunger from the syringe body; and
translating the plunger within the syringe body sufficient to generate pressures exceeding 30 atmospheres (ATM) within the syringe body.

21. The method of claim 20, wherein threads on the plunger are configured to engage and disengage with threads on the syringe body at a first angle and wherein the plunger is configured to couple and decouple from the syringe body at a second angle, wherein the first angle is greater than the second angle.
